SEQUENCE LISTING (44 sequences)

SEQ ID NO: 1 - DNA, 21 bp, synthetic construct
  source 1..21: mol_type = other DNA
gacctgagcctgagagctctg

SEQ ID NO: 2 - DNA, 190 bp, synthetic construct
  source 1..190: mol_type = other DNA
agcagcagtgcctcctccccacctaagaaggagcaccatcatcaccaccatcactcagagtccccaaaggcgcccgtgccactgctcccaccccatcagcccccctgggcctcaggacttgagcagcagcatctgcaaagaagagaagatgccccgaggaggctcactggaaagcgatggctgccccaag

SEQ ID NO: 3 - DNA, 23 bp, synthetic construct
  source 1..23: mol_type = other DNA
cccctgagcctcaggacttgagc

SEQ ID NO: 4 - DNA, 23 bp, synthetic construct
  source 1..23: mol_type = other DNA
ctgagcctcaggacttgagcagc

SEQ ID NO: 5 - DNA, 23 bp, synthetic construct
  source 1..23: mol_type = other DNA
cctgagcctcaggacttgagcag

SEQ ID NO: 6 - protein, 17 aa, Homo sapiens
HSESPKAPVPLLPPLPP

SEQ ID NO: 7 - protein, 17 aa, synthetic construct
HSESTKAPMPLLPPLSP

SEQ ID NO: 8 - protein, 18 aa, Homo sapiens
HSESPKAPVPLLPPHQPP

SEQ ID NO: 9 - protein, 18 aa, synthetic construct
HSESTKAPMPLLPPHQPP

SEQ ID NO: 10 - DNA, 102 bp, Homo sapiens
  source 1..102: mol_type = genomic DNA
cactcagagtccccaaaggcccccgtgccactgctcccacccctgcccccacctccacctgagcccgagagctccgaggaccccaccagcccccctgagccc

SEQ ID NO: 11 - protein, 34 aa, Homo sapiens
HSESPKAPVPLLPPLPPPPPEPESSEDPTSPPEP

SEQ ID NO: 12 - DNA, 102 bp, Homo sapiens; identical to SEQ ID NO: 10
  source 1..102: mol_type = genomic DNA

SEQ ID NO: 13 - protein, 34 aa, Homo sapiens; identical to SEQ ID NO: 11

SEQ ID NO: 14 - protein, 34 aa, Homo sapiens; identical to SEQ ID NO: 11
  VARIANT 1: note = Amino acid residue may be Pro or Tyr
  VARIANT 2: note = Amino acid residue may be Pro
  VARIANT 4: note = Amino acid residue may be Pro or Phe
  VARIANT 5: note = Amino acid residue may be Ser
  VARIANT 6: note = Amino acid residue may be Glu
  VARIANT 7: note = Amino acid residue may be Val, Ser, Thr, Gly or Asp
  VARIANT 8: note = Amino acid residue may be Ser or Leu
  VARIANT 9: note = Amino acid residue may be Met
  VARIANT 10: note = Amino acid residue may be Ser
  VARIANT 12: note = Amino acid residue may be Phe
  VARIANT 13: note = Amino acid residue may be Ser, Thr or Leu
  VARIANT 14: note = Amino acid residue may be Ser, Thr, Leu or His
  VARIANT 15: note = Amino acid residue my be Pro
  VARIANT 16: note = Amino acid residue may be Leu or Thr
  VARIANT 17: note = Amino acid residue may be Ser, Leu or Ala
  VARIANT 18: note = Amino acid residue may be Ser, His or Arg
  VARIANT 21: note = Amino acid residue may be Ala
  VARIANT 22: note = Amino acid residue may be Ser or Ala
  VARIANT 23: note = Amino acid residue may be Lys
  VARIANT 25: note = Amino acid residue may be Phe
  VARIANT 26: note = Amino acid residue may be Lys
  VARIANT 27: note = Amino acid residue may be Tyr
  VARIANT 28: note = Amino acid residue may be Leu or Ser
  VARIANT 29: note = Amino acid residue may be Ile
  VARIANT 30: note = Amino acid residue may be Gly, Asn or Arg
  VARIANT 31: note = Amino acid residue may be Leu, Ser, Thr, Ala or His
  VARIANT 34: note = Amino acid residue may be Leu, Ser or Ala
  VARIANT 19: note = Amino acid residue may be Ser
  VARIANT 32: note = Amino acid residue may be Ser, Leu or Ala

SEQ ID NO: 15 - DNA, 102 bp, Homo sapiens
  source 1..102: mol_type = genomic DNA
aaggcccccgtgccactgctcccacccctgcccccacctccacctgagcccgagagctccgaggaccccaccagcccccctgagccccaggacttgagcagc

SEQ ID NO: 16 - protein, 34 aa, Homo sapiens
KAPVPLLPPLPPPPPEPESSEDPTSPPEPQDLSS

SEQ ID NO: 17 - protein, 33 aa, Homo sapiens
  SITE 32: note = Deleted amino acid
RPPCHCSHPCPHLHLSPRAPRTPPAPLSPRTXA

SEQ ID NO: 18 - protein, 34 aa, Homo sapiens
  SITE 15: note = Deleted amino acid
  SITE 27: note = Deleted amino acid
GPRATAPTPAPTSTXARELRGPHQPPXAPGLEQQ

SEQ ID NO: 19 - protein, 4 aa, Homo sapiens
HQPP

SEQ ID NO: 20 - protein, 15 aa, Homo sapiens
HSESPKAPVPPHQPP

SEQ ID NO: 21 - protein, 20 aa, Homo sapiens
HSESPKAPVPLLRGPHQPPX

SEQ ID NO: 22 - protein, 14 aa, Homo sapiens
HSESPKAPVPLLPP

SEQ ID NO: 23 - protein, 18 aa, Homo sapiens
HSESPKAPVPLLPPHQPP

SEQ ID NO: 24 - protein, 17 aa, Homo sapiens
HSESPKAPVPLLPPQPP

SEQ ID NO: 25 - protein, 22 aa, Homo sapiens
HSESPKAPVPLLPPLRGPHQPP

SEQ ID NO: 26 - protein, 22 aa, Homo sapiens
HSESPKAPVPLLPPLRGPHQPP

SEQ ID NO: 27 - protein, 18 aa, Homo sapiens
HSESPKAPVPLLPPLQPP

SEQ ID NO: 28 - protein, 23 aa, Homo sapiens
HSESPKAPVPLLPPLPRGPHQPP

SEQ ID NO: 29 - protein, 20 aa, Homo sapiens
HSESPKAPVPLLPPLPHQPP

SEQ ID NO: 30 - protein, 17 aa, Homo sapiens
HSESPKAPVPLLPPLPP

SEQ ID NO: 31 - protein, 23 aa, Homo sapiens
HSESPKAPVPLLPPLPPGPHQPP

SEQ ID NO: 32 - protein, 21 aa, Homo sapiens
HSESPKAPVPLLPPAPLSPRT

SEQ ID NO: 33 - protein, 19 aa, Homo sapiens
HSESPKAPVPLLPPLSPRT

SEQ ID NO: 34 - protein, 34 aa, Homo sapiens
HSESPKAPVPLLPPLLHLSPRAPRTPPAPLSPRT

SEQ ID NO: 35 - protein, 27 aa, Homo sapiens
HSESPKAPVPLLPPLPRTPPAPLSPRT

SEQ ID NO: 36 - protein, 34 aa, Homo sapiens; identical to SEQ ID NO: 34

SEQ ID NO: 37 - protein, 25 aa, Homo sapiens
HSESPKAPVPLLPPLPPPAPLSPRT

SEQ ID NO: 38 - protein, 36 aa, Homo sapiens
HSESPKAPVPLLPPLPPPHLSPRAPRTPPAPLSPRT

SEQ ID NO: 39 - protein, 35 aa, Homo sapiens
HSESPKAPVPLLPPLPPPPSPRAPRTPPAPLSPRT

SEQ ID NO: 40 - protein, 35 aa, Homo sapiens
HSESPKAPVPLLPPLPPPPPEPEPRTPPAPLSPRT

SEQ ID NO: 41 - DNA, 114 bp, synthetic construct
  source 1..114: mol_type = other DNA
ccccaccagcccccctgagcctcaggacttgagcagcagcatctgcaaagaagagaagatgccccgaggaggctcactggaaagcgatggctgccccaaggagccagctaagac

SEQ ID NO: 42 - protein, 5 aa, synthetic construct
PHQPP

SEQ ID NO: 43 - protein, 35 aa, synthetic construct
HQPPWASGLEQQHLQRREDAPRRLTGKRWLPQGAS

SEQ ID NO: 44 - DNA, 33 bp, synthetic construct
  source 1..33: mol_type = other DNA
caccagcccccctgagcctcaggacttgagcag

(19) United States
(12) Patent Application Publication
(10) Pub. No.: US 2026/0325675 A1
(43) Pub. Date: Oct. 22, 2026

(54) Rett Syndrome Therapy

(71) Applicants: The University Court of the University of Edinburgh, Edinburgh (GB); The General Hospital Corporation, Boston, MA (US)

(72) Inventors: Benjamin KLEINSTIVER, Boston, MA (US); Jacqueline GUY, Edinburgh (GB); Adrian BIRD, Edinburgh (GB)

(21) Appl. No.: 19/109,672

(22) PCT Filed: Sep. 7, 2023
(86) PCT No.: PCT/GB2023/052315

§ 371 Date: Mar. 7, 2025

Related U.S. Application Data
(60) Provisional application No. 63/375,008, filed on Sep. 8, 2022.

(51) Int. Cl. A61K 48/00; C12N 15/11; C12N 15/86

(52) U.S. Cl. CPC A61K 48/005; C12N 15/111; C12N 15/86; C12N 2750/14143

(57) ABSTRACT

The present disclosure relates to compositions for use in the treatment of a class of Rett syndrome mutations, namely C-terminal deletions, comprising a base editor to alter a stop codon in a mutant MECP2 gene. This alteration does not return the gene to its wild-type (WT) form, but re-establishes normal levels of a version which is functionally equivalent to a wild, type version of the MeCP2 protein.

### FIELD OF THE DISCLOSURE

The present disclosure relates to compositions for use in the treatment of a class of Rett syndrome mutations, namely C-terminal deletions, comprising a base editor to alter a stop codon in a mutant MECP2 gene. This alteration does not return the gene to its wild-type (WT) form, but re-establishes normal levels of a version which is functionally equivalent to a wild-type version of the MeCP2 protein.

### BACKGROUND OF THE DISCLOSURE

Rett Syndrome (RTT) is a severe neurological disorder predominantly caused by mutations in the X-linked gene MECP2. Loss of MeCP2 function has the most profound effect in the nervous system, with minimal phenotypic consequences in other tissues ((Ross et al. 2016). Because of its association with intellectual disability in RTT and other disorders, the MECP2 gene is frequently screened for mutations in clinical cases of developmental delay. This has defined many amino acid changes as RTT-causing or as relatively benign or neutral variants that do not cause RTT (Krishnaraj et al 2017, and Karczewski, et al. 2020).

RTT-like symptoms in Mecp2-null mice can be rescued by restoring MeCP2 expression, suggesting that the disorder may be curable. Most RTT-causing mutations disrupt two domains, which are required for normal MeCP2 function; however, there are mutations, which affect the region C-terminal to these domains. These mutations cause RTT by dramatically reducing MeCP2 protein levels (Guy et al. 2018). Interestingly, mice lacking this C-terminal region, which express normal levels of MeCP2 protein, do not have RTT-like symptoms, indicating that this region is dispensable for normal MeCP2 function.

### SUMMARY OF THE DISCLOSURE

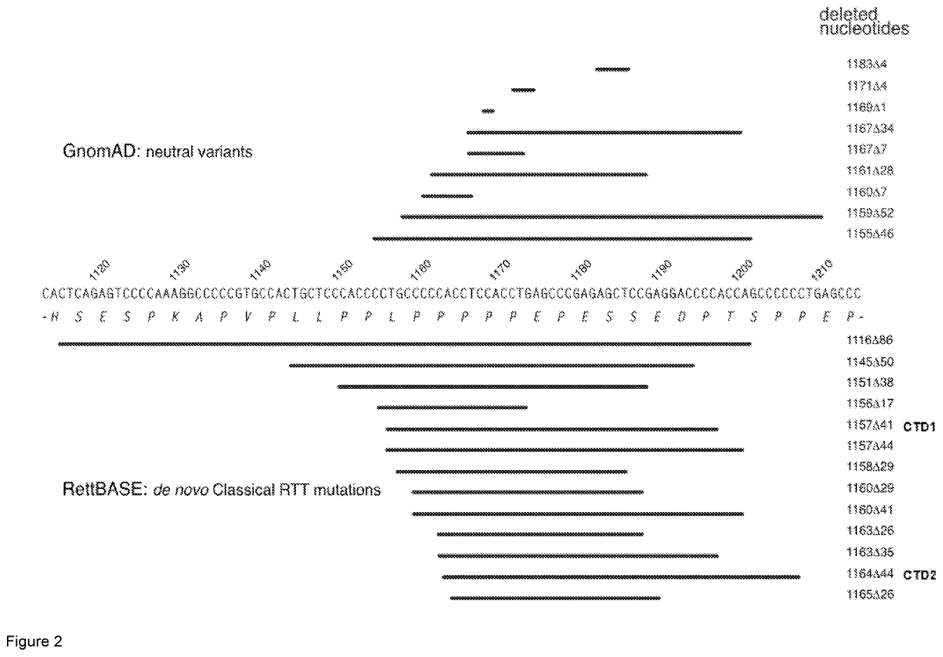

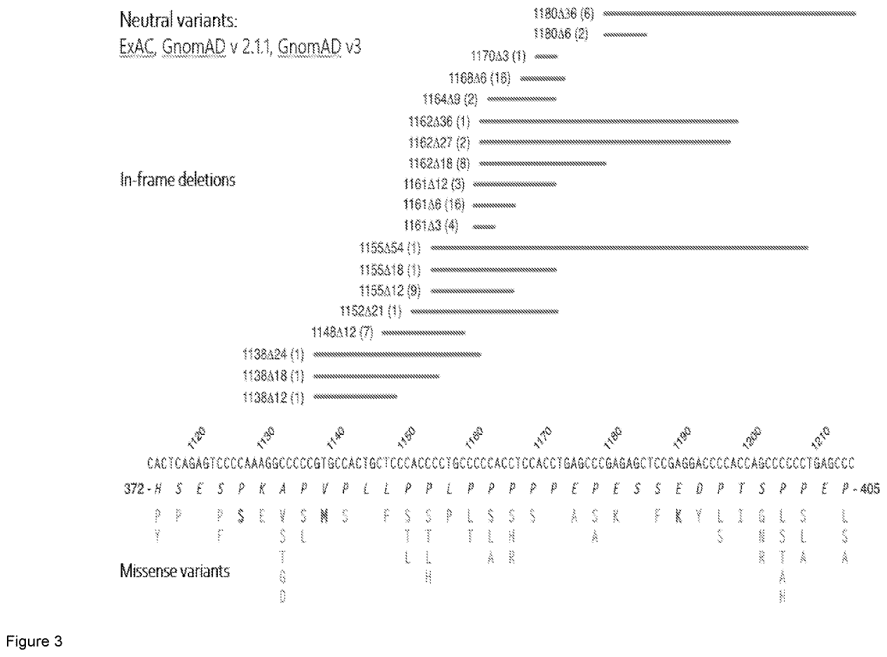

A heterogeneous group of frameshifting C-terminal deletions (CTDs) account for ~10% of RTT-causing mutations. They occur within a deletion-prone region of MECP2 from approximately c.1110-1210 (numbered based on e2 isoform) (Bebbington et al, 2010). The inventors have found that the outcome of deletions in this region is dependent on the resulting reading frame. In-frame deletions which remove a portion of the DNA in this region, but still maintain the WT reading frame are present in large-scale sequencing databases, which exclude individuals with severe paediatric disease, indicating that they are non-pathogenic neutral variants (see FIG. 3). The same is true for frameshifting deletions which shift to frame(n+1), and end in the amino acid sequence -Ser-Pro-Arg-Thr-Stop. However, further RTT-causing frameshifting deletions which appear in RettBASE (a database which lists mutations found in RTT patients), shift to frame(n+2) and end in -Pro-Pro-Stop (see FIG. 2).

The present disclosure is based on an initial hypothesis by the inventors that for RTT subjects, with a deletion in exon 4 of MECP2, which result in a shift to frame(n+2), it is the presence of the two prolines before a stop codon which disrupt translational termination, resulting in loss of MeCP2 protein and mRNA in a process similar to nonsense-mediated decay. Based on this, the inventors predicted that changing the stop codon following the two prolines to one coding an amino acid, leading to further translation to the next stop codon, may prevent the loss of MeCP2 protein that is pathogenic in this class of mutations.

Thus, in a first aspect the present disclosure provides a base editing construct for use in a method of treating Rett Syndrome in a subject, wherein the subject comprises a mutant MECP2 gene comprising a C-terminal deletion which results in a translation frameshift (n+2) and expression of a truncated MECP2 gene ending -Pro-Pro-Stop, wherein the construct is capable of editing the stop codon in order to permit translational read-through.

As mentioned above, the present inventors observed, from sequence information obtained from subjects with Rett syndrome, that certain C terminal deletions, approximately c.1110-1210 (numbered based on e2 isoform), result in a +2 frameshift and the generation of a truncated MECP2 protein, which ends -Pro-Pro-Stop (PPX). The nucleic acid sequence encoding this is CCCCCCTGA. In order to avoid translational termination occurring at the TGA stop codon, the present invention describes editing the adenine base in the TGA stop codon to another base, such as guanine, or optionally cytosine, or thymine to result in a codon other than a stop codon. For example, altering the adenine base in the TGA stop codon, to a guanine, gives a TGG (tryptophan) codon. As described in further detail herein, the present inventors have produced a mouse with a knock-in mutation, which exactly models this A to G change in the CTD1 mouse model previously described in Guy et al (2108). In contrast to CTD1 mice, this new model shows no RTT-like phenotypes and 100% survival up to one year, demonstrating that this approach may be used to develop a therapy for this class of RTT mutations.

In accordance with the invention, one suitable base editing method involves using a single-guide RNA (sgRNA) to target an adenine base editor (ABE) to the target adenine of the TGA stop codon. Due to the nature of the local DNA sequence, it may be appropriate to use ABEs which recognise non-canonical PAM sequences (see Walton et al, 2020, for example). Further more active PAM-variant ABEs are described in, for example, Richter (2020), Walton (2020), Gaudelli (2020) and Chen (2022), the entire contents of which are hereby incorporated by way of reference. The inventors have observed that the guide sequence is present in (almost) all CTD RTT-alleles. Thus, the therapy should be applicable to this whole class of mutations. The edited gene will not produce wild-type protein, but will encode a truncation that in the work exemplified herein retains the functions of native MeCP2.

The present disclosure provides a number of advantages in relation to other therapeutic strategies for treating RTT:

Avoids Issues with Gene Dosage

One of the main therapeutic strategies for treating RTT, which is currently being explored, is gene therapy, which involves viral delivery of exogenous MECP2. However, a major challenge facing this form of gene therapy is that overexpression of MeCP2 results in other neurological disorders, such as MECP2 duplication syndrome. Therefore, it is difficult to determine a dose that is therapeutic, whilst avoiding MeCP2 overexpression. By editing endogenous MECP2 using a base editor, such as ABE, the gene remains under the control of its endogenous regulatory elements, thus circumventing any issues with gene dosage.

### Permanent Correction

Other therapeutic approaches for RTT include RNA editing approaches or use of read-through compounds for nonsense mutations. A limitation of these strategies is that they will require repeat dosing in order to maintain levels of corrected mRNA. A base editing technique such as using an ABE on the other hand, will result in permanent correction of the gene, and prove therapeutic once a sufficient number of cells have been modified.

### Only Requires One DNA Edit (Doesn't Rely on Double-Stranded DNA Breaks (DSBs), HDR and Co-Delivery of a Repair Template)

Ideally, gene editing would simply revert the mutation back to WT, which in theory could be done using homology-directed repair (HDR) of Cas9-induced double strand breaks (DSBs), by supplying an exogenous DNA repair template. In that case the strategy would be highly useful for ex vivo approaches where the edited cells can be expanded in culture first before returning them to the body, or if a population of dividing cells in which there is a selective advantage for edited cells over unedited cells is being targeted. Unfortunately, this approach is unsuitable for RTT because the target population is post-mitotic neurons, in which HDR levels are low and there is no means of selection. The present strategy avoids this problem by relying solely on the single step of editing the endogenous sequence, rather than editing/inserting a new sequence. DSBs introduced as part of the HDR repair process carry a higher risk of undesirable mutagenic events than base editing.

One sgRNA (or a Set of sgRNAs) can be Used for all CTD Mutations

RTT C-terminal deletions are a heterogeneous set of mutations, with many individual deletions seen only once or a small number of times. This strategy targets the problematic sequence that is thought to be present in the whole class, thus making the therapy applicable to around 10% of RTT patients, comparable with the most commonly detected missense mutations.

Functional Domains should be Unaffected

The two critical functional domains, the MBD (methylated DNA binding domain; a.a.78-162) and the NID (NCoR1/2 interaction domain; a.a.301-309) are unaffected by CTD mutations. ExAC and GnomAD data shows that the C-terminal deletion-prone region is very tolerant of missense mutations and in-frame deletions.

The discovery and widespread implementation of the CRISPR/Cas system has dramatically expanded the toolbox for genome engineering and has revolutionized the future prospects of basic biological research and medicine. The recent development of adenine base editors by fusion of a deaminase domain to Cas9 enables guide RNA (gRNA)-targeted single nucleotide deamination for A:T base pair conversion to G:C using adenine base editors within a specific target window. Base editing has been broadly demonstrated with high efficiency in a range of species, including human zygotes.

Various engineered base editors with improved DNA editing efficiencies have been developed. Reference is made, for example, to U.S. Patent Publication No. 2018/0073012, published Mar. 15, 2018; U.S. Patent Publication No. 2017/0121693, published May 4, 2017; International Publication No. WO 2017/070633, published Apr. 27, 2017; and U.S. Patent Publication No. 2015/0166980, published Jun. 18, 2015, U.S. Pat. No. 9,840,699, issued Dec. 12, 2017; and U.S. Pat. No. 10,077,453, issued Sep. 18, 2018, each of which are incorporated herein in their entireties. Base editors (BEs) may be fusions of a Cas (“CRISPR-associated”) domain and a nucleobase (or “base”) modification domain (e.g., a natural or evolved deaminase, such as an adenosine deaminase domain). In some cases, base editors may also include proteins or domains that affect cellular DNA repair processes to increase the efficiency and/or stability of the resulting single-nucleotide change.

Base editors generally contain a catalytically impaired Cas9 domain fused to a nucleobase modification domain. The Cas9 domain directs the nucleobase modification domain to directly convert one base to another at a guide RNA-programmed target site. Two classes of base editors have been developed to date: Cytosine base editors (CBEs), which convert C*G to TA, and adenine base editors (ABEs), which convert A-T to G*C. The present disclosure is directed to the use of ABEs

ABEs (see for example Gaudelli et al. 2017 and Richter et al 2020, to which the skilled reader is directed and the entire contents of which are herein incorporated by way of reference) are especially useful for the study and correction of pathogenic alleles, as nearly half of pathogenic point mutations in principle can be corrected by converting an A-T base pair to a G*C base pair. Many of the ABEs reported to date include a single polypeptide chain containing a heterodimer of a wild-type E. coli TadA monomer (ecTadA, or TadA) that plays a structural role during base editing and a laboratory-evolved E. coli TadA monomer TadA7.10 (also referred to herein as “TadA*”) that catalyzes deoxyadenosine deamination, and a Cas9 (D10A) nickase. Wild type E. coli TadA acts as a homodimer to deaminate an adenosine located in a tRNA anticodon loop, generating inosine (1). Although early ABE variants required a heterodimeric TadA containing an N-terminal wild-type TadA monomer for maximal activity, later work showed that later ABE variants have comparable activity with and without the wild-type TadA monomer.

Guide RNA-dependent off-target base editing has been reduced through strategies including installation of mutations that increase DNA specificity into the Cas9 component of base editors, adding 5′ guanosine nucleotides to the sgRNA, or delivery of the base editor as a ribonucleoprotein complex (RNP). Guide RNA-independent off-target editing can arise from binding of the deaminase domain of a base editor to C or A bases in a Cas9-independent manner.

The ABEs for use in accordance with the present disclosure may comprise fusion proteins comprising a nucleic acid DNA binding protein (or napDNAbp) domain and an adenosine deaminase domain. The napDNAbp domain may comprise a Cas9 protein, or a variant thereof, e.g., a Cas9 nickase or a Cas9 nickase with altered PAM specificity. The adenosine deaminase domain may comprise one or more adenosine deaminases. In certain teachings, the adenosine deaminase domain comprises a dimer of a first and second adenosine deaminase. The dimer may be a heterodimer, comprising a first adenosine deaminase that is different from a second adenosine deaminase. The first adenosine deaminase may be positioned N-terminal to the second adenosine deaminase. In various embodiments, the one or more adenosine deaminases are connected by a linker (e.g., a peptide linker).

Suitable ABEs may be capable of preserving DNA editing efficiency, and in some embodiments demonstrate improved DNA editing efficiencies, relative to existing adenine base editors, such as ABE7.10—see, for example WO2020214842, the entire contents of which are hereby incorporated. WO2020214842 describes ABEs, which exhibit reduced off-target editing effects while retaining high on-target editing efficiencies, as well as more recent ABEs described in Richter (2020), Walton (2020), Gaudelli (2020) and Chen (2022).

Suitable ABEs may be compatible with a variety of Cas homologs, including small-sized, circularly permuted, and evolved Cas homologs.

The present disclosure includes the use of compositions comprising an ABE, such as an ABE with reduced off-target effects, such as reduced RNA editing effects e.g., fusion proteins comprising an nCas9 domain and an adenosine deaminase domain (e.g., a heterodimer of a first and second adenosine deaminase), and one or more guide RNAs, e.g., a single-guide RNA (“sgRNA”).

The present disclosure further teaches nucleic acid molecules encoding and/or expressing the adenine base editors as described herein, and the adenosine deaminase domains thereof, as well as expression vectors or constructs for expressing the adenine base editors described herein and a gRNA (e.g. sgRNA), cells (such as host cells) comprising said nucleic acid molecules and expression vectors, and one or more gRNAs (e.g. sgRNA), and compositions for delivering and/or administering nucleic acid molecules for expression in cells of RTT subjects with the described deletion in exon 4 of MECP2, which result in a shift to frame(n+2). The nucleic acid sequences may be codon-optimized for expression in human cells, using techniques well-known to those skilled in the art.

The present specification further teaches complexes comprising the adenine base editors described herein and a gRNA (e.g. sgRNA) bound to the Cas9 domain of the fusion protein. The guide RNA may be 50-150, such as 90-100 or 80-110 nucleotides in length and comprise a sequence of at least 10, at least 15, or at least 20 or 25 contiguous nucleotides that is complementary to a target nucleotide sequence. Typically, the sequence is no longer than 30 or 35 nucleotides in length.

The disclosure further includes kits for expressing and/or transducing cells (such as host cells) with an expression construct encoding the fusion protein and gRNA. There is further taught kits for administration of expressed fusion protein and expressed gRNA (e.g. sgRNA) molecules to a host cell. The disclosure further teaches host cells stably or transiently expressing the fusion protein and gRNA (e.g. sgRNA), or a complex thereof.

As the present disclosure is directed to the treatment of RTT due to specific C terminal deletions that result in a frameshift (n+2) and a truncated MECP2 sequence ending -Pro-Pro-Stop, the present teaching may further include first screening RTT subjects for such C-terminal frameshift (n+2) deletion mutations, in order to identify subjects suitable for treatment in accordance with the present invention. The skilled reader is well aware of suitable screening techniques, which may be employed, including nucleic acid sequencing of a subject's MDCP2 gene sequence, PCR and other amplification techniques, hybridisation techniques using suitable probes etc.

Methods are also taught for editing a mutant MECP2 gene, as described herein, e.g., a single nucleobase within the mutant MECP2 gene, with an adenine base editor described herein. Such methods involve transducing (e.g., via transfection) cells with a plurality of complexes each comprising a fusion protein (e.g., a fusion protein comprising a Cas9 nickase (nCas9) domain and an adenosine deaminase domain) and a gRNA (e.g. sgRNA) molecule. In certain embodiments, the methods involve the transfection of one or more nucleic acid constructs (e.g., plasmids, phagemids, or viral vectors) that each (or together) encode the components of a complex of fusion protein and gRNA (e.g. sgRNA) molecule. In other teachings, the methods disclosed herein may involve the introduction into cells of a complex comprising a fusion protein and gRNA (e.g. sgRNA) molecule that has been expressed and cloned outside of these cells. In accordance with the invention, delivery to neurons in the brain, using a viral vector, such as an adeno-associated virus (AAV) vector (e.g. AAV9), is most appropriate. However, nucleic acid encoding suitable base editors may be too big, to fit into and be expressed by a single viral vector. Thus, in one teaching, as described herein, the base editor may be encoded in two or more separate parts, which can be reconstituted into the full-length molecule by protein splicing. This is facilitated by the addition of split intein sequences adjacent to the sequences to be joined (see Chen (2020, for example).

In some embodiments, methods of treating RTT due to a mutant MECP2 gene as described herein, using the disclosed base editors are provided. The methods described herein may comprise treating a subject having or at risk of developing RTT, comprising administering (in an effective amount) to the subject a fusion protein as described herein, a complex as described herein, a polynucleotide as described herein, a vector as described herein, or a pharmaceutical composition as described herein.

### Definitions

As used herein and in the claims, the singular forms “a”, “an,” and “the” include the singular and the plural reference unless the context clearly indicates otherwise. Thus, for example, a reference to “an agent” includes a single agent and a plurality of such agents.

As used herein, the term “adenosine deaminase domain” refers to a domain within a fusion protein comprising one or more adenosine deaminases. For instance, an adenosine deaminase domain may comprise a heterodimer of a first adenosine deaminase and a second deaminase domain, connected by a linker, or a single engineered adenosine deaminase domain.

“Base editing” refers to genome editing technology that involves the conversion of a specific nucleic acid base into another at a targeted genomic locus. In certain embodiments, this can be achieved without requiring double-stranded DNA breaks (DSB), or single stranded breaks (i.e. nicking). Other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g. typically 0.1% to 5%), with the major genome editing products being indels. In order to increase the efficiency of gene correction without simultaneously introducing random indels, the CRISPR/Cas9 system has been modified to directly convert one DNA base into another without DSB formation. See, Komor, A. C., et al, Programmable editing of a target base in genomic DNA without double-stranded DNA cleavage. Nature 533, 420-424 (2016), the entire contents of which is incorporated by reference herein.

“Base editing construct” refers to a system comprising a suitable enzyme and optionally a nucleic acid capable of binding a target nucleic acid, for use in base editing.

“Adenine base editor” (or “ABE”). This type of editor converts an A:T Watson-Crick nucleobase pair to a G:C Watson-Crick nucleobase pair. Because the corresponding Watson-Crick paired bases are also interchanged as a result of the conversion, this category of base editor may also be referred to as a thymine base editor (or “TBE”).

The term “base editor” (or “BE”) as used herein, refers to an agent comprising a polypeptide that is capable of making a modification to a base (e.g., A, T, C, G, or U) within a nucleic acid sequence (e.g., DNA or RNA). In some embodiments, the base editor is capable of deaminating a base within a nucleic acid such as a base within a DNA molecule.

In the case of an adenine base editor, the base editor is capable of deaminating an adenine (A) in DNA. Such base editors may include a nucleic acid programmable DNA binding protein (napDNAbp) fused to an adenosine deaminase. Some base editors include CRISPR-mediated fusion proteins that are utilized in the base editing methods described herein. In some embodiments, the base editor comprises a nuclease-inactive Cas9 (dCas9) fused to a deaminase which binds a nucleic acid in a guide RNA-programmed manner via the formation of an R-loop, but does not cleave the nucleic acid. For example, the dCas9 domain of the fusion protein may include a D10A and a H840A mutation (which renders Cas9 capable of cleaving only one strand of a nucleic acid duplex), as described in WO 2017/070632 and is incorporated herein by reference in its entirety. The DNA cleavage domain of S. pyogenes Cas9 includes two subdomains, the HNH nuclease subdomain and the RuvCI subdomain. The HNH subdomain cleaves the strand complementary to the gRNA (the “targeted strand”, or the strand in which editing or deamination occurs), whereas the RuvCI subdomain cleaves the non-complementary strand containing the PAM sequence (the “non-edited strand”). The RuvCI mutant D10A generates a nick in the targeted strand, while the HNH mutant H840A generates a nick on the non-edited strand (see Jinek et al, Science, 337:816-821(2012); Qi et al, Cell. 28; 152(5): 1173-83 (2013)).

The term “base editor” encompasses the CRISPR-mediated fusion proteins utilized in the multiplexed base editing methods described herein as well as any base editor known or described in the art at the time of this filing or developed in the future. Reference is made to Rees & Liu, Base editing: precision chemistry on the genome and transcriptome of living cells, Nat. Rev. Genet. 2018; 19(12):770-788; as well as U.S. Patent Publication No. 2018/0073012; U.S. Patent Publication No. 2017/0121693; International Publication No. WO2017/070633; U.S. Patent Publication No. 2015/0166980; International Publication No. WO 2017/070633; International Publication No. WO 2018/027078; International Publication No. WO 2019/079347; International Publication No. WO2019/226593; U.S. Patent Publication No. 2015/0166980; U.S. Pat. No. 10,077,453; International Publication No. WO 2019/023680; International Publication No. WO 2018/0176009; International publication No. WO2020051360; International Publication No. WO2020102659; International publication No. WO202086908; and International publication No. WO2020214842, the contents of each of which are incorporated herein by reference in their entireties.

The term “Cas9” or “Cas9 nuclease” or “Cas9 domain” refers to a CRISPR-associated protein 9, or variant thereof, and embraces any naturally occurring Cas9 from any organism, any naturally-occurring Cas9 equivalent or fragment thereof, any Cas9 homolog, ortholog, or paralog from any organism, and any variant of a Cas9, naturally-occurring or engineered. The term Cas9 thus extends to compact Cas9 variants, such as Nme2 variants, which have been developed (see Erdaki et al, 2019)

The term Cas9 is not meant to be particularly limiting and may be referred to as a “Cas9 or variant thereof.” Exemplary Cas9 proteins are described herein and also described in the art. The present disclosure is unlimited with regard to the particular Cas9 that is employed in the CRISPR-mediated fusion proteins utilized in the disclosure.

In some embodiments, proteins comprising Cas9 or fragments thereof are referred to as “Cas9 variants.” A Cas9 variant shares homology to Cas9, or a fragment thereof. Cas9 variants include functional fragments of Cas9. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9.

In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

As used herein, the term “dCas9” refers to a nuclease-inactive Cas9 or nuclease-dead Cas9, or a variant thereof, and embraces any naturally occurring dCas9 from any organism, any naturally-occurring dCas9 equivalent or functional fragment thereof, any dCas9 homolog, ortholog, or paralog from any organism, and any variant of a dCas9, naturally-occurring or engineered. The term dCas9 is not meant to be particularly limiting and may be referred to as a “dCas9 or variant thereof.” Exemplary dCas9 proteins and method for making dCas9 proteins are further described herein and/or are described in the art and are incorporated herein by reference. Any suitable mutation which inactivates both Cas9 endonucleases, such as D10A and H840A mutations in the wild-type S. pyogenes Cas9 amino acid sequence, or D10A and N580A mutations in the wild-type S. aureus Cas9 amino acid sequence, may be used to form the dCas9.

As used herein, the term “nCas9” or “Cas9 nickase” refers to a Cas9 or a variant thereof, which cleaves or nicks only one of the strands of a target cut site thereby introducing a nick in a double strand DNA molecule rather than creating a double strand break. This can be achieved by introducing appropriate mutations in a wild-type Cas9, which inactivates one of the two endonuclease activities of the Cas9. Any suitable mutation which inactivates one Cas9 endonuclease activity but leaves the other intact is contemplated, such as one of D10A or H840A mutations in the wild-type S. pyogenes Cas9 amino acid sequence, or a D10A mutation in the wild-type S. aureus Cas9 amino acid sequence, may be used to form the nCas9.

“CRISPR” is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively constitute, along with an array of CRISPR-associated proteins (including Cas9 and homologs thereof) and CRISPR-associated RNA, a prokaryotic immune defence system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (me) and a Cas9 protein. The tracrRNA serves as a guide for ribonuclease 3-aided processing of pre-crRNA. Subsequently, Cas9/crRNA/tracrRNA endonucleolytically cleaves linear or circular nucleic acid target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) may be engineered so as to incorporate embodiments of both the crRNA and tracrRNA into a single RNA species—the guide RNA. See, e.g., Jinek M., et al., Science 337:816-821(2012), the entire contents of which is herein incorporated by reference. Cas9 recognizes a short motif in the CRISPR repeat sequences (the PAM or protospacer adjacent motif) to help distinguish self, versus non-self. CRISPR biology, as well as Cas9 nuclease sequences and structures are well known to those of skill in the art (see, e.g., “Complete genome sequence of an MI strain of Streptococcus pyogenes.” Ferretti J. J., et al, Proc. Natl. Acad. Sci. U.S.A. 98:4658-4663(2001); “CRISPR RNA maturation by trans-encoded small RNA and host factor RNase Ill.” Deltcheva E., et al, Nature 471:602-607(2011); and “A programmable dual-RNA-guided DNA endonuclease in adaptive bacterial immunity.” Jinek M., et al, Science 337:816-821(2012), the entire contents of each of which are incorporated herein by reference). Cas9 orthologs have been described in various species, including, but not limited to, S. pyogenes, S. thermophiles, C. ulcerans, S. diphtheria, S. syrphidicola, P. intermedia, S. taiwanense, S. iniae, B. baltica, P. torquis, S. thermophilus, L. innocua, C. jejuni and N. meningitidis. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737, the entire contents of which are incorporated herein by reference. Other relevant teachings, to which the skilled reader is directed and the entire contents of which are hereby incorporated by way of reference include Kleinstiver (2016, Slaymaker (2015) and Vakulskas (2018).

The term “deaminase” or “deaminase domain” refers to a protein or enzyme that catalyzes a deamination reaction. In accordance with the disclosure, the deaminase is an adenosine deaminase, which catalyzes the hydrolytic deamination of adenine or adenosine. In some embodiments, the adenosine deaminase catalyzes the hydrolytic deamination of adenosine in deoxyribonucleic acid (DNA) to inosine (and thus the conversion of adenine base to hypoxanthine base). The deaminases provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

Adenosine deaminases (e.g. engineered adenosine deaminases, evolved adenosine deaminases) provided herein may be enzymes that convert adenosine (A) to inosine (I) in DNA or RNA. Such adenosine deaminase can lead to an A:T to G:C base pair conversion. In some embodiments, the deaminase is a variant of a naturally-occurring deaminase from an organism. In some embodiments, the deaminase does not occur in nature. For example, in some embodiments, the deaminase is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

In some embodiments, the adenosine deaminase is derived from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. Reference is made to U.S. Patent Publication No. 2018/0073012, the entire contents of which is incorporated herein by reference.

As used herein, the term “DNA binding protein” or “DNA binding protein domain” refers to any protein that localizes to and binds a specific target DNA nucleotide sequence (e.g. a gene locus of a genome). This term embraces RNA-programmable proteins, which associate (e.g. form a complex) with one or more nucleic acid molecules (i.e., which includes, for example, guide RNA in the case of Cas systems) that direct or otherwise program the protein to localize to a specific target nucleotide sequence (e.g., DNA sequence) that is complementary to the one or more nucleic acid molecules (or a portion or region thereof) associated with the protein. Exemplary RNA-programmable proteins are CRISPR-Cas9 proteins, as well as Cas9 equivalents, homologs, orthologs, or paralogs, whether naturally occurring or non-naturally occurring (e.g. engineered or modified), and may include a Cas9 equivalent from any type of CRISPR system (e.g. type II, V, VI), including Casl2a (a type-V CRISPR-Cas system) (formerly known as Cpf1), C2cl (a type V CRISPR-Cas system), C2c2 (a type VI CRISPR-Cas system), C2c3 (a type V CRISPR-Cas system), a GeoCas9, a CjCas9, a Casl2b, a Casl2g, a Casl2h, a Casl2i, a Casl3b, a Casl3c, a Casl3d, a Cas14, a Csn2, an xCas9, an SpCas9-NG, a Cas9-KKH, a circularly permuted Cas9, an Argonaute (Ago), a SmacCas9, or a Spy-macCas9. Further Cas-equivalents are described in Makarova et al, “C2c2 is a single-component programmable RNA-guided RNA-targeting CRISPR effector,” Science 2016; 353(6299), the contents of which are incorporated herein by reference.

The term “DNA editing efficiency,” as used herein, refers to the number or proportion of intended base pairs that are edited. For example, if a base editor edits 10% of the base pairs that it is intended to target (e.g., within a cell or within a population of cells), then the base editor can be described as being 10% efficient. Some aspects of editing efficiency embrace the modification (e.g. deamination) of a specific nucleotide within DNA, without generating a large number or percentage of insertions or deletions (i.e., indels). It is generally accepted that editing while generating less than 5% indels (as measured over total target nucleotide substrates) is high editing efficiency. The generation of more than 20% indels is generally accepted as poor or low editing efficiency. Indel formation may be measured by techniques known in the art, including high-throughput screening of sequencing reads.

The term “off-target editing frequency,” as used herein, refers to the number or proportion of unintended base pairs, e.g. DNA base pairs that are edited. On-target and off-target editing frequencies may be measured by the methods and assays described herein, further in view of techniques known in the art, including high-throughput sequencing reads. As used herein, high-throughput sequencing involves the hybridization of nucleic acid primers (e.g., DNA primers) with complementarity to nucleic acid (e.g., DNA) regions just upstream or downstream of the target sequence or off-target sequence of interest. Because the DNA target sequence and the Cas9-independent off-target sequences are known a priori in the methods disclosed herein, nucleic acid primers with sufficient complementarity to regions upstream or downstream of the target sequence and Cas9-independent off-target sequences of interest may be designed using techniques known in the art, such as the PhusionU PCR kit (Fife Technologies), Phusion HS II kit (Fife Technologies), and Illumina MiSeq kit. Since many of the Cas9-dependent off-target sites have high sequence identity to the target site of interest, nucleic acid primers with sufficient complementarity to regions upstream or downstream of the Cas9-dependent off-target site may likewise be designed using techniques and kits known in the art. These kits make use of polymerase chain reaction (PCR) amplification, which produces amplicons as intermediate products. The target and off-target sequences may comprise genomic loci that further comprise protospacers and PAMs.

Accordingly, the term “amplicons,” as used herein, may refer to nucleic acid molecules that constitute the aggregates of genomic loci, protospacers and PAMs. High-throughput sequencing techniques used herein may further include Sanger sequencing and/or whole genome sequencing (WGS).

The terms “RNA editing activity,” “RNA editing effects” and “RNA off-target editing,” as used herein, refer to the introduction of modifications (e.g. deaminations) to nucleotides within cellular RNA, e.g. messenger RNA (mRNA). An important goal of DNA base editing efficiency is the modification (e.g. deamination) of a specific nucleotide within DNA, without introducing modifications of similar nucleotides within RNA. RNA editing effects are “low” or “reduced” when a detected mutation is introduced into RNA molecules at a frequency of 0.3% or less.

For reference, the ABEmax base editor introduces edits into RNA at a frequency of about 0.50%. RNA editing effects are “low” or “reduced” when a mutation is detected at a magnitude that is less than about 70,000 edits within an analyzed mRNA transcriptome. The number of RNA edits may be measured by techniques known in the art, including high-throughput screening of sequencing reads and RNA-seq. The effects of RNA editing on the function of a protein translated from the edited mRNA transcript may be predicted by use of the SIFT (“Sorting Intolerant from Tolerant”) algorithm, which bases predictions on sequence homology and the physical properties of amino acids.

The term “on-target editing,” as used herein, refers to the introduction of intended modifications (e.g., deaminations) to nucleotides (e.g., adenine) in a target sequence, such as using the base editors described herein. The term “off-target DNA editing,” as used herein, refers to the introduction of unintended modifications (e.g. deaminations) to nucleotides (e.g. adenine) in a sequence outside the canonical base editor binding window (i.e., from one protospacer position to another, typically 2 to 8 nucleotides long). Off-target DNA editing can result from weak or non-specific binding of the gRNA sequence to the target sequence. Exemplary teachings, the entire contents of which are hereby incorporated by way of reference, describing ways of reducing off-target editing may be found in Grunwald (2019) and Rees (2019).

The term “effective amount”, as used herein, refers to an amount of a biologically active agent that is sufficient to elicit a desired biological response. For example, in some embodiments, an effective amount of a composition may refer to the amount of the composition that is sufficient to edit a target site of a nucleotide sequence, e.g. a genome. In some embodiments, an effective amount of a composition provided herein, e.g. of a composition comprising a nuclease-inactive Cas9 domain, a deaminase domain, a gRNA and optionally a growth factor and anti-apoptotic factor, may refer to the amount of the composition that is sufficient to induce editing of a target site specifically bound and edited by the fusion protein. In some embodiments, an effective amount of a composition provided herein may refer to the amount of the composition sufficient to induce editing having the following characteristics: >50% product purity, <5% indels, and an editing window of 2-8 nucleotides. As will be appreciated by the skilled artisan, the effective amount of an agent, e.g. a composition or a fusion protein-gRNA complex, may vary depending on various factors as, for example, on the desired biological response, e.g. on the specific allele, genome, or target site to be edited, on the cell or tissue being targeted, and on the agent being used.

The term “evolved base editor” or “evolved base editor variant” refers to a base editor formed as a result of mutagenizing a reference or starting-point base editor. The term refers to embodiments in which the nucleobase modification domain is evolved or a separate domain is evolved. Mutagenizing a reference or starting-point base editor may comprise mutagenizing an adenosine deaminase. Amino acid sequence variations may include one or more mutated residues within the amino acid sequence of a reference base editor, e.g., as a result of a change in the nucleotide sequence encoding the base editor that results in a change in the codon at any particular position in the coding sequence, the deletion of one or more amino acids (e.g., a truncated protein), the insertion of one or more amino acids, or any combination of the foregoing. The evolved base editor may include variants in one or more components or domains of the base editor (e.g., variants introduced into one or more adenosine deaminases).

The term “fusion protein” as used herein refers to a hybrid polypeptide, which comprises protein domains from at least two proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “host cell,” as used herein, refers to a cell that can host, replicate, and transfer a nucleic acid or vector as discussed herein. In embodiments where the vector is a viral vector, a suitable host cell is a cell that may be infected by the viral vector, can replicate it, and can package it into viral particles that can infect fresh host cells. A cell can host a viral vector if it supports expression of genes of viral vector, replication of the viral genome, and/or the generation of viral particles. One criterion to determine whether a cell is a suitable host cell for a given viral vector is to determine whether the cell can support the viral life cycle of a wild-type viral genome that the viral vector is derived from. For example, if the viral vector is a modified M13 phage genome, as provided in some embodiments described herein, then a suitable host cell would be any cell that can support the wild-type M13 phage life cycle. Suitable host cells for viral vectors useful in continuous evolution processes are well known to those of skill in the art, and the disclosure is not limited in this respect. In some embodiments, the viral vector is a phage and the host cell is a bacterial cell. In some embodiments, the host cell is an E. coli cell. Suitable

E. coli host strains will be apparent to those of skill in the art, and include, but are not limited to, New England Biolabs (NEB) Turbo, ToplOF′, DH12S, ER2738, ER2267, and XLI-Blue MRF′. These strain names are art recognized and the genotype of these strains has been well characterized. The term “fresh,” as used herein interchangeably with the terms “non-infected” or “uninfected” in the context of host cells, refers to a host cell that has not been infected by a viral vector comprising a gene of interest as used in a continuous evolution process provided herein. A fresh host cell can, however, have been infected by a viral vector unrelated to the vector to be evolved or by a vector of the same or a similar type but not carrying the gene of interest.

In some embodiments, the host cell is a prokaryotic cell, for example, a bacterial cell. In some embodiments, the host cell is an E. coli cell. In some embodiments, the host cell is a eukaryotic cell, for example, a yeast cell, an insect cell, or a mammalian cell. The type of host cell, will, of course, depend on the viral vector employed, and suitable host cell/vector combinations will be readily apparent to those of skill in the art.

As Rett syndrome is a neurological disorder that affects the way the brain functions, the host cell may be a neurological cell, such as a neuron, e.g. an excitatory or inhibitory neuron, or glial cells such as astrocytes and microglia.

The term “linker,” as used herein, refers to a chemical group or a molecule linking two molecules or domains, e.g. dCas9 and a deaminase. Typically, the linker is positioned between, or flanked by, two groups, molecules, or other domains and connected to each one via a covalent bond, thus connecting the two. In some embodiments, the linker is an amino acid or a plurality of amino acids (e.g. a peptide or protein). In some embodiments, the linker is an organic molecule, group, polymer, or chemical domain. Chemical groups include, but are not limited to, disulfide, hydrazone, and azide domains. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, the linker is an XTEN linker.

As used herein, the term “low toxicity” refers to the maintenance of a viability above 60% in a population of cells following application of a base editing method or administration of a composition disclosed herein. The term may also refer to prevention of apoptosis (cell death) in a population of cells of more than 40%. For instance, a genome editing method that leads to less than 30% (e.g. 25%, 20%, 15%, 10%, or 5%) cell death exhibits low toxicity. Cell toxicity may be assessed by an appropriate staining assay, e.g. Annexin V and propidium iodide staining assays, and subsequent flow cytometry (e.g. FACS).

The term “mutation,” as used herein, refers to a substitution of a residue within a sequence, e.g., a nucleic acid or amino acid sequence, with another residue; a deletion or insertion of one or more residues within a sequence; or a substitution of a residue within a sequence of a genome in a subject to be corrected. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is a result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome, which results from a mutation in the gene for the connective tissue protein called fibrillin.

“Translational read-though” in the context of the present disclosure, is the consequence editing the third base of the TGA stop codon, such that the resulting codon no longer encodes a stop codon and translation can continue until the next stop codon occurs.

The terms “non-naturally occurring” or “engineered” are used interchangeably and indicate the involvement of the hand of man. These terms, when referring to nucleic acid molecules or polypeptides (e.g. deaminases) mean that the nucleic acid molecule or the polypeptide is at least substantially free from at least one other component with which they are naturally associated in nature and/or as found in nature (e.g. an amino acid sequence not found in nature).

The term “nucleic acid,” as used herein, refers to RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g. a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g. analogs having other than a phosphodiester backbone. Nucleic acids may be purified from natural sources, produced using recombinant expression systems and optionally purified, chemically synthesized, etc. Where appropriate, e.g. in the case of chemically synthesized molecules, nucleic acids may comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g. 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, inosinedenosine, 8-oxoguanosine, 0(6)-methylguanine, and 2-thiocytidine); chemically modified bases; biologically modified bases (e.g. methylated bases); intercalated bases; modified sugars (e.g. 2′-fluororibose, ribose, 2′-deoxyribose, arabinose, and hexose); and/or modified phosphate groups (e.g. phosphorothioates and 5′-N-phosphoramidite linkages).

As used herein to modify guide RNA molecules, the term “backbone” refers to the component of the guide RNA that comprises the core region, also known as the crRNA/tracrRNA. The backbone is separate from the guide sequence, or spacer, region of the guide RNA, which has complementarity to a protospacer of a nucleic acid molecule.

The term “nucleic acid programmable DNA binding protein (napDNAbp)” refers to any protein that may associate (e.g., form a complex) with one or more nucleic acid molecules (i.e., which may broadly be referred to as a “napDNAbp-programming nucleic acid molecule” and includes, for example, guide RNA in the case of Cas systems) which direct or otherwise program the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. This term napDNAbp embraces CRISPR-Cas9 proteins, as well as Cas9 equivalents, homologs, orthologs, or paralogs, whether naturally occurring or non-naturally occurring (e.g., engineered or modified), and may include a Cas9 equivalent from any type of CRISPR system (e.g., type II, V, VI), including Casl2a (a type-V CRISPR-Cas system) (formerly known as Cpfl), C2cl (a type V CRISPR-Cas system), C2c2 (a type VI CRISPR-Cas system), C2c3 (a type V CRISPR-Cas system), a GeoCas9, a CjCas9, a Cas 12b, a Casl2g, a Casl2h, a Casl2i, a Casl3b, a Casl3c, a Casl3d, a Cas14, a Csn2, an xCas9, an SpCas9-NG, a Cas9-KKH, a circularly permuted Cas9, an Argonaute (Ago), a SmacCas9, or a Spy-macCas9. The napDNAbp may be a Cas9 domain that comprises a nuclease active Cas9 domain, a nuclease inactive Cas9 (dCas9) domain, or a Cas9 nickase (nCas9) domain. Further Cas equivalents are described in Makarova et al., “C2c2 is a single-component programmable RNA-guided RNA-targeting CRISPR effector,” Science 2016; 353 (6299), the contents of which are incorporated herein by reference. However, the nucleic acid programmable DNA binding protein (napDNAbp) that may be used in connection with this disclosure are not limited to CRISPR-Cas systems. The claimed invention embraces any such programmable protein, such as the Argonaute protein from Natronobacterium gregoryi (NgAgo) which may also be used for DNA-guided genome editing. NgAgo-guide DNA system does not require a PAM sequence or guide RNA molecules, which means genome editing can be performed simply by the expression of generic NgAgo protein and introduction of synthetic oligonucleotides on any genomic sequence. See Gao et al, DNA-guided genome editing using the Natronobacterium gregoryi Argonaute. Nature Biotechnology 2016; 34(7):768-73, which is incorporated herein by reference.

In some embodiments, the napDNAbp is a RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in Figure IE of Jinek et al, Science 337:816-821(2012), the entire contents of which is incorporated herein by reference. Other examples of gRNAs (e.g., those including domain 2) can be found in U.S. Pat. No. 9,340,799, entitled “mRNA-Sensing Switchable gRNAs,” and WO 2015/035136 and entitled “Delivery System For Functional Nucleases,” the entire contents of each are herein incorporated by reference. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csnl) from Streptococcus pyogenes (see, e.g., “Complete genome sequence of an MI strain of Streptococcus pyogenes.” Ferretti J. J. et al., Proc. Natl. Acad. Sci. U.S.A. 98:4658-4663(2001); “CRISPR RNA maturation by trans-encoded small RNA and host factor RNase Ill.” Deltcheva E. et al, Nature 471:602-607(2011); and “A programmable dual-RNA-guided DNA endonuclease in adaptive bacterial immunity.” Jinek M. et al, Science 337:816-821(2012), the entire contents of each of which are incorporated herein by reference.

The napDNAbp nucleases (e.g., Cas9) use RNA:DNA hybridization to target DNA cleavage sites, these proteins are able to be targeted, in principle, to any sequence specified by the guide RNA. Methods of using napDNAbp nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature Biotechnology 31, 227-229 (2013); Jinek, M. et al. RNA-programmed genome editing in human cells. eLife 2, e00471 (2013); Dicarlo, J. E. et al., Genome engineering in Saccharomyces cerevisiae using CRISPR-Cas systems. Nucleic Acid Res. (2013); Jiang, W. et al. RNA-guided editing of bacterial genomes using CRISPR-Cas systems. Nature Biotechnology 31, 233-239 (2013); the entire contents of each of which are incorporated herein by reference).

The term “napDNAbp-programming nucleic acid molecule” or equivalently “guide sequence” refers the one or more nucleic acid molecules which associate with and direct or otherwise program a napDNAbp protein to localize to a specific target nucleotide sequence (e.g., a gene locus of a genome) that is complementary to the one or more nucleic acid molecules (or a portion or region thereof) associated with the protein, thereby causing the napDNAbp protein to bind to the nucleotide sequence at the specific target site. A non limiting example is a guide RNA of a Cas protein of a CRISPR-Cas genome editing system.

The term “promoter” is art-recognized and refers to a nucleic acid molecule with a sequence recognized by the cellular transcription machinery and able to initiate transcription of a downstream gene. A promoter may be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity.

Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the present disclosure, which is not limited in this respect. In various embodiments, the disclosure provides vectors with appropriate promoters for driving expression of the nucleic acid sequences encoding the fusion proteins (or one or more individual components thereof).

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g. a genetically engineered non-human subject. The subject may be of either sex and at any stage of development.

The term “target site” or “target nucleic acid” refers to a sequence within a nucleic acid molecule that is edited by a fusion protein (e.g. a dCas9-deaminase fusion protein provided herein). The target site further refers to the sequence within a nucleic acid molecule to which a complex of the fusion protein and gRNA binds.

The terms “treatment,” “treat,” and “treating,” refer to a clinical intervention aimed to reverse, alleviate, delay the onset of, or inhibit the progress of a disease, disorder, or condition, or one or more symptoms thereof, as described herein. As used herein, the terms “treatment,” “treat,” and “treating” refer to a clinical intervention aimed to reverse, alleviate, delay the onset of, or inhibit the progress of a disease, disorder, or condition, or one or more symptoms thereof, as described herein. In some embodiments, treatment may be administered after one or more symptoms have developed and/or after a disease has been diagnosed. In other embodiments, treatment may be administered in the absence of symptoms, e.g. to prevent or delay onset of a symptom or inhibit onset or progression of a disease. For example, treatment may be administered to a susceptible individual prior to the onset of symptoms (e.g. in light of a history of symptoms and/or in light of genetic or other susceptibility factors). Treatment may also be continued after symptoms have resolved, for example, to prevent or delay their prevention or recurrence.

As used herein, the term variant” refers to a protein having characteristics that deviate from what occurs in nature that retains at least one functional i.e. binding, interaction, or enzymatic ability and/or therapeutic property thereof. A “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. For instance, a variant of Cas9 may comprise a Cas9 that has one or more changes in amino acid residues as compared to a wild type Cas9 amino acid sequence. As another example, a variant of a deaminase may comprise a deaminase that has one or more changes in amino acid residues as compared to a wild type deaminase amino acid sequence, e.g. following ancestral sequence reconstruction of the deaminase. These changes include chemical modifications, including substitutions of different amino acid residues truncations, covalent additions (e.g. of a tag), and any other mutations. This term also embraces fragments of a wild type protein.

The level or degree of which the property is retained may be reduced relative to the wild type protein but is typically the same or similar in kind. Generally, variants are overall very similar, and in many regions, identical to the amino acid sequence of the protein described herein. A skilled artisan will appreciate how to make and use variants that maintain all, or at least some, of a functional ability or property.

The variant proteins may comprise, or alternatively consist of, an amino acid sequence which is at least 80%, 85%, 90%, 95%, 96%, 97%, 98%, 99%, or 100%, identical to, for example, the amino acid sequence of a wild-type protein, or any protein provided herein (e.g. Cas9 protein, fusion protein, and fusion protein protein). Further polypeptides provided in the disclosure are encoded by polynucleotides which hybridize to the complement of a nucleic acid molecule encoding a protein such as a Cas9 protein under stringent hybridization conditions (e.g. hybridization to filter bound DNA in 6× Sodium chloride/Sodium citrate (SSC) at about 45 degrees Celsius, followed by one or more washes in 0.2.times.SSC, 0.1% SDS at about 50-65 degrees Celsius), under highly stringent conditions (e.g. hybridization to filter bound DNA in 6× sodium chloride/S odium citrate (SSC) at about 45 degrees Celsius, followed by one or more washes in 0.IxSSC, 0.2% SDS at about 68 degrees Celsius), or under other stringent hybridization conditions which are known to those of skill in the art (see, for example, Ausubel, F. M. et al, eds., 1989 Current Protocol in Molecular Biology, Green publishing associates, Inc., and John Wiley & Sons Inc., New York, at pp. 6.3.1-6.3.6 and 2.10.3).

By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98%, or 99% identical to, for instance, the amino acid sequence of a protein such as a Cas9 protein, can be determined conventionally using known computer programs.

A preferred method for determining the best overall match between a query sequence (a sequence of the present disclosure) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=I, Joining Penalty=20, Randomization Group Length=0, Cutoff Score=I, Window Size=sequence length, Gap Penalty=5, Gap Size Penalty=0.05, Window Size=500 or the length of the subject amino acid sequence, whichever is shorter. If the subject sequence is shorter than the query sequence due to N- or C-terminal deletions, not because of internal deletions, a manual correction must be made to the results.

This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present disclosure. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

As used herein, the term “wild type” is a term of the art understood by skilled persons and means the typical form of an organism, strain, gene or characteristic as it occurs in nature as distinguished from mutant or variant forms.

The present invention will now be further described by way of example and with reference to the Figures, which show:

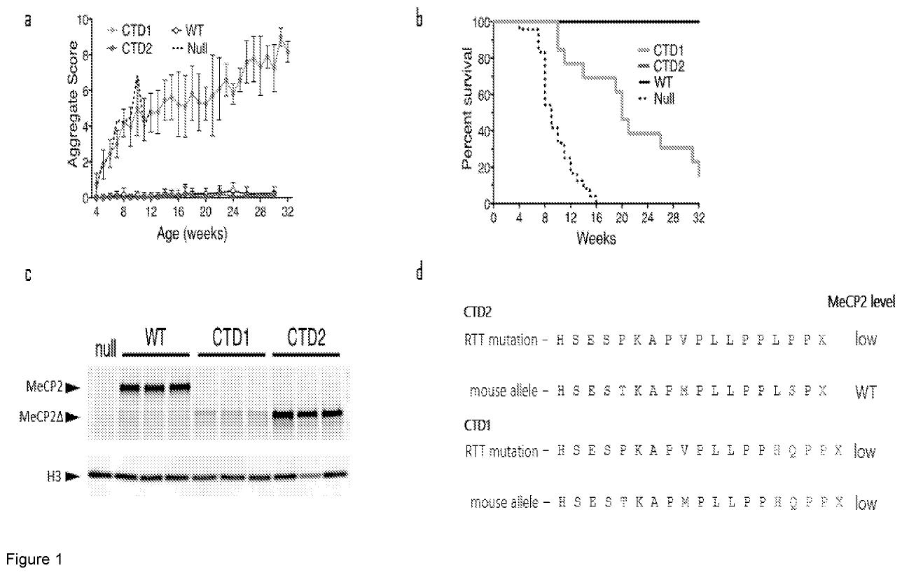

FIG. 1. CTD1 and CTD2 knock-in mice unexpectedly show different phenotypes.

CTD1 and CTD2 mouse alleles were intended to model the two most common RTT CTD patient mutations. (a) Male mice hemizygous for the CTD1 mutation show the expected RTT-like phenotypes, similar to Mecp2 null animals, whereas CTD2 mice appear indistinguishable from wild-type littermates. (b) CTD1 mice show reduced survival (median age at death 20 weeks). CTD2 mice have 100% survival at one year, as WT littermates. (c) CTD2 mice express truncated MeCP2 at WT levels (whole brain protein from 6 week-old mice), whereas CTD1 mice have reduced levels of MeCP2 protein in brain. (d) Due to differences in amino acid sequence between mouse and humans, the mouse CTD2 allele encodes -SPX at the C-terminus rather than -PPX found in patients with the equivalent mutation. Both mouse and patient alleles for CTD1 end with -PPX;

FIG. 2. Human neutral variants and patient frameshifting mutations appear to overlap

The human MECP2 deletion-prone region is shown with neutral variants above and high-confidence Rett mutations below (black lines indicate the deleted nucleotides). Numbering of the DNA and amino acid sequence is that of the E2 isoform. The patient mutations, which were modelled in mice, CTD1 and CTD2, are indicated;

FIG. 3. Missense mutations and in-frame deletions in the CTD region

The human MECP2 deletion-prone region is shown with neutral variants from the ExAC and GnomAD databases: in-frame deletion variants are shown above and missense mutations below. Green lines indicate deleted nucleotides. Letters below indicated missense amino acid changes, with those found in more than 10 individuals indicated in purple and more than 50 individuals in bold purple. Numbering of the DNA and amino acid sequence is that of the E2 isoform;

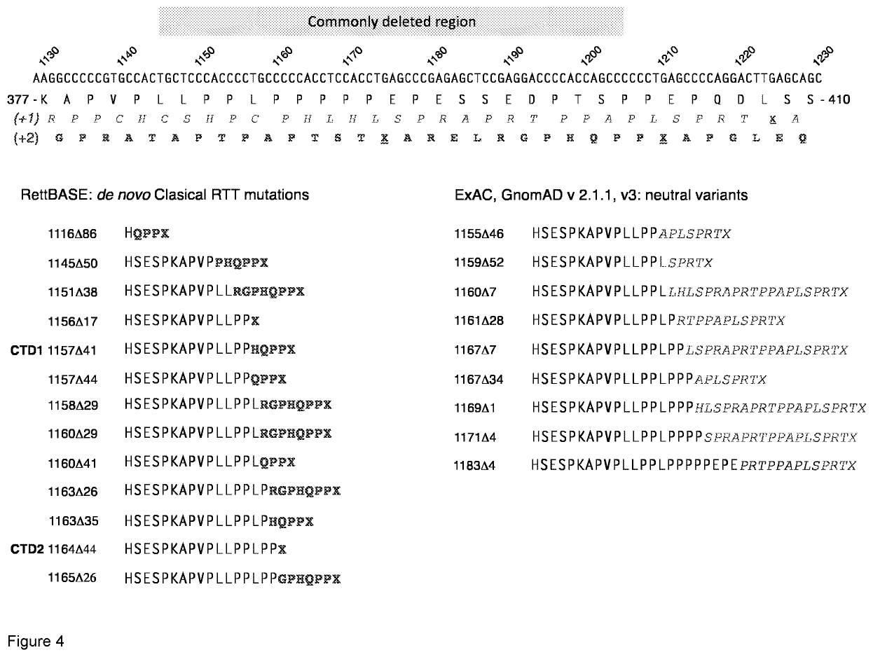

FIG. 4. Pathogenicity of deletions correlates with translation frame

The human MECP2 commonly deleted region is shown with the possible amino acid sequences arising from frameshifting deletions. The C-terminal amino acid sequences of MeCP2 from Rett mutations and neutral variants with C-terminal deletions are shown, with the frameshifted amino acids using bold or italic typeface according to their frame. STOP codons X, are underlined;

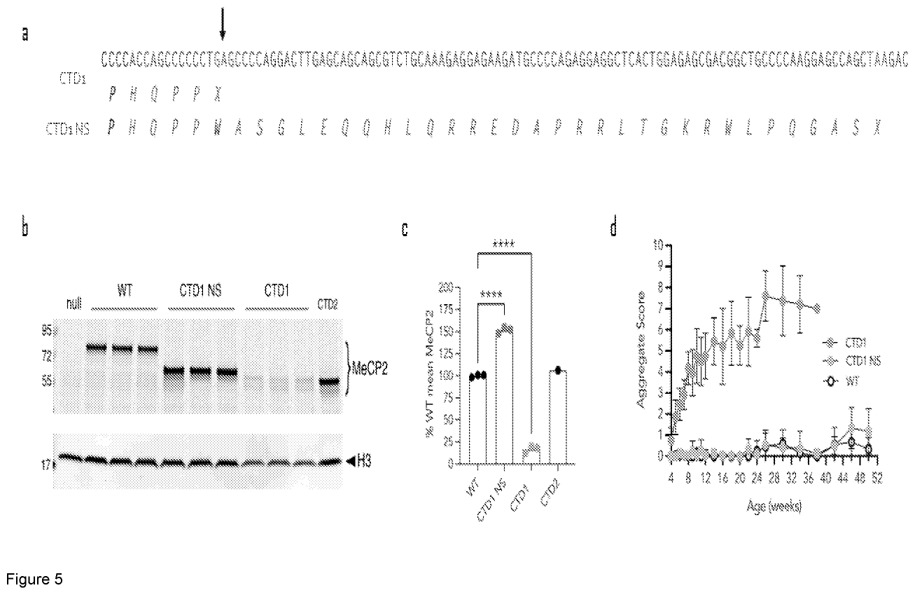

FIG. 5. CTD1 NS knock-in mouse replaces the TGA stop by a TGG tryptophan codon

(a) partial DNA sequence of mouse Mecp2 CTD1 allele showing the stop codon in red, with a black arrow indicating the adenine base which is to be edited. This A is changed to G in the CTD1 NS mouse allele. The resulting C-terminal amino acid sequence is shown. (b) Reduced levels of MeCP2 in CTD1 mouse brain are restored to approximately WT levels in CTD1 NS mice where the stop codon is replaced by tryptophan (assayed in whole brain protein from 6 week old mice). (c) Quantification of western blot in (b). (d) Male mice hemizygous for the CTD1 mutation show RTT-like phenotypes similar to Mecp2 null animals, while CTD1 NS mice are indistinguishable from wild-type littermates;

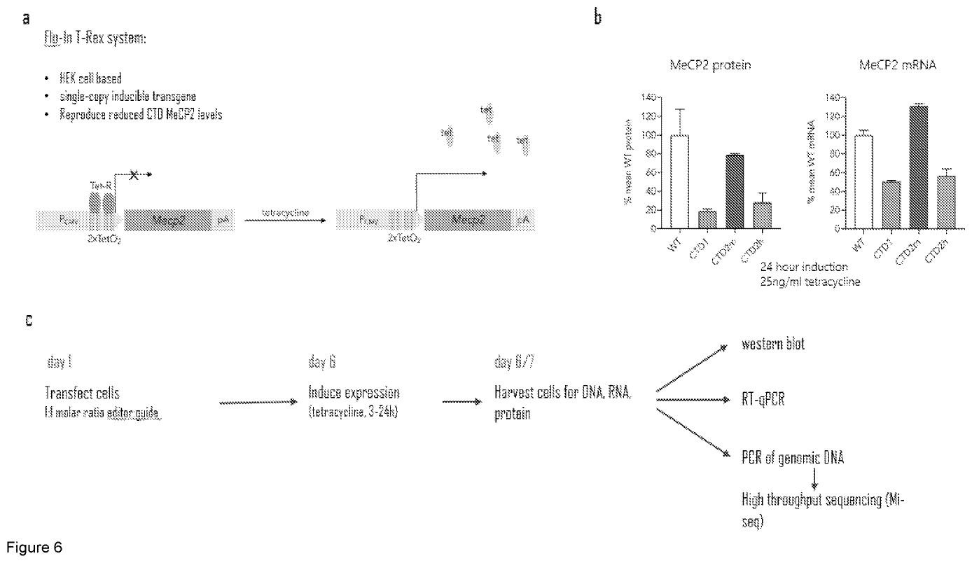

FIG. 6. A cell culture system to test base editing reagents

(a) Overview of the Flp-In T-Rex system showing the Mecp2 cDNA transgene under the control of a tetracycline-inducible promoter. (b) Single copy Mecp2 cDNA transgenes reproduce the levels of mutant MeCP2 protein and mRNA seen from knock-in mouse alleles. (c) Scheme of work for transient transfection experiments to test ABE/guide RNA combinations;

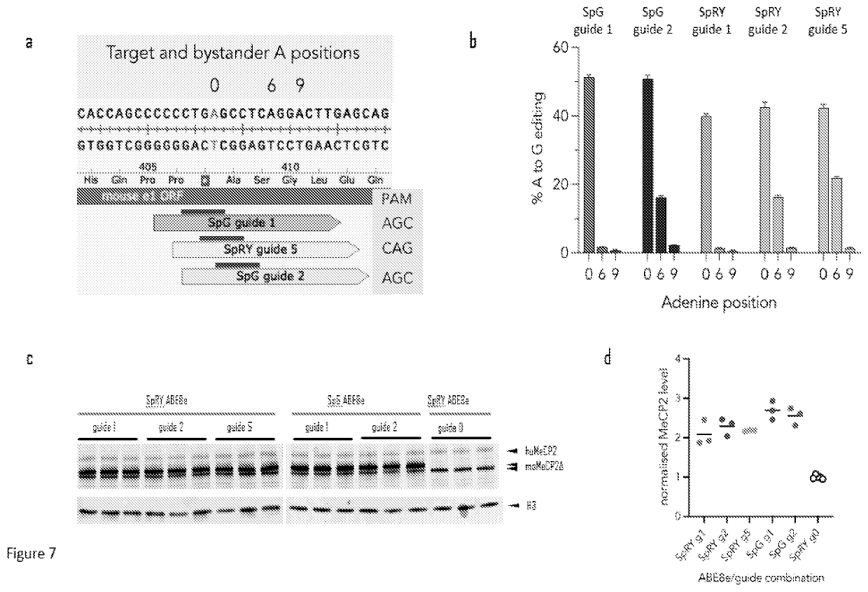

FIG. 7. A to G editing in cultured human cells expressing a MeCP2 CTD1 transgene

(a) Nucleotide sequence of the region surrounding the target adenine (A) in the CTD1 Mecp2 transgene. The target A is indicated as position 0 and bystander As within the guide RNA sequence as 6 and 9. Guide RNA sequences are shown below, with their optimal editing windows indicated by a bar. Guides 1 and 2 can be utilized bySpG and SpRY ABE8es, and guide 5 by SpRY ABE8e only. (b) Quantification of adenine base editing at target and bystander sites in the CTD1 Mecp2 transgene following transfection with different combinations of ABE and guide RNA expression constructs. High throughput sequencing of PCR amplicons from triplicate transfections of each plasmid combination are shown. (c) Western blot of protein from the ABE/guide combinations shown in (b). The level of truncated MeCP2 protein is increased after successful editing. Edited CTD1 MeCP2 protein runs above unedited protein due to the extended C-terminal tail. Endogenous full-length human MeCP2 from T-Rex cells is also present. Cells were harvested at day 6 after 24 hours of CTD1 Mecp2 transgene induction. (d) Quantification of data in panel (c). Total truncated MeCP2 level (edited+unedited CTD1 MeCP2) was normalised to the histone H3 loading control. Expression levels are normalised to the mean empty guide value (g0) which was set to 1. Individual data points and mean values are shown;

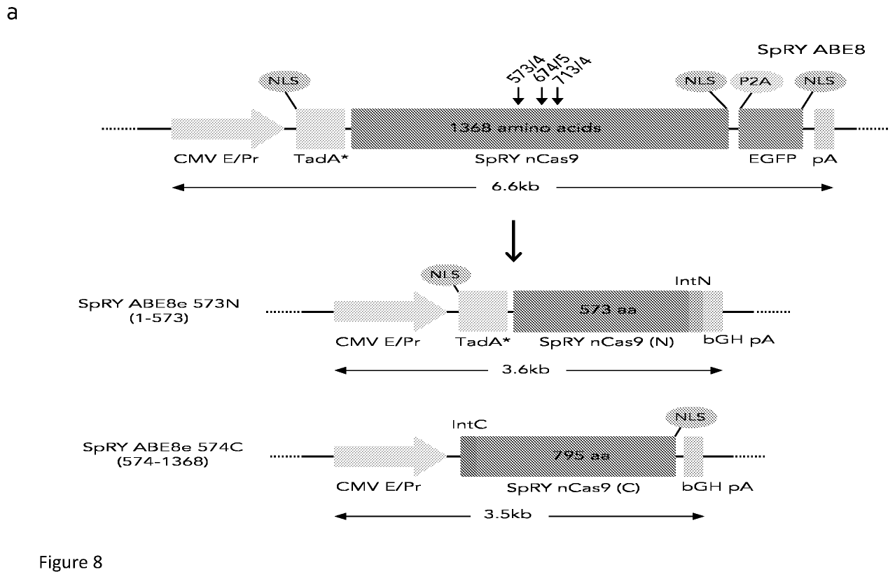

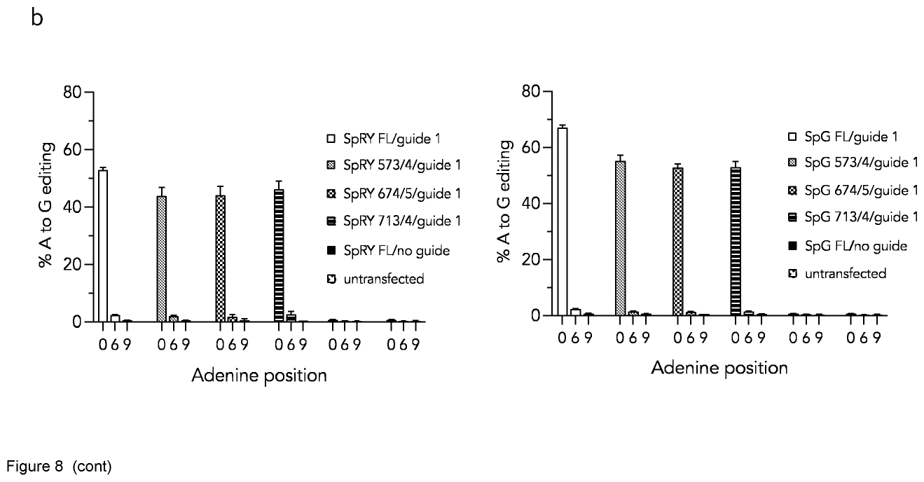

FIG. 8. A to G editing with reconstituted split ABE expression constructs

(a) Diagram of full-length SpRY ABE8e expression construct showing 3 split sites in the SpCas9 component. An example of a split pair of expression constructs is shown below. These constructs include split intein sequences from the DnaB gene from the thermophilic bacterium Rhodothermus marinus. Addition of these sequences to the two portions of ABE8e results in protein splicing when the two polypeptides are co-expressed in the same cell, thus reconstituting a full-length ABE. (b) Quantification of A to G base editing following transfection of split ABE pairs and guide RNA expression constructs into CTD1 Mecp2 Flp-In T-Rex cells. Editing at the target A and two bystander As is shown, as in FIG. 7. SpG and SpRY ABE8e variants were tested using 3 different split sites.

Referring to FIG. 1, Figure panels a-c are taken from Guy et al (2018). Hum. Mol. Genet., 27, 2531-2545. All information relating to the experimental details, materials and methods used to generate the data are included in that publication.

This data shows that the C-terminus of the protein is not required for MeCP2 function (CTD2 mouse) and that the CTD1 mutation is pathogenic due to greatly reduced levels of MeCP2 protein.

The ExAC and GnomAD sequencing databases (comprising exome and genomic sequence data from individuals in the general population, known not to suffer with severe neurological disease) were searched for deletions in the C-terminal region of MECP2. These deletions (see FIG. 2) are depicted above the nucleotide and amino acid sequence of the region. The information was extracted from ExAC and GnomAD v 2.1.1 and v3 in October 2019 and from GnomAD in May 2023, when 3 additional frameshifting deletions were found GnomAD v3, namely 1167del7, 1167del34 and 1183del4.

Below the sequence (see FIG. 2) are shown deletions found in the RettBASE database of RTT patient mutations. To exclude non-pathogenic mutations in MECP2 (there are a number of these in RettBASE), only cases which had classical Rett syndrome, and where the mutation was not present in either parent, were studied. FIG. 3 shows in-frame deletion and missense neutral variants extracted from the ExAC and GnomAD databases in October 2019. This data highlights that the amino acids in this region of MeCP2 are not important for the function of the protein.

The three possible reading frames of the C-terminal deletion-prone region are shown in FIG. 4: the normal reading frame in standard type, +1 in italic and +2 in bold. Below the C-terminal amino acid sequence of the frame-shifting deletions in FIG. 2 are shown. The correct amino acids before the frame-shift are shown in standard type, with those after formatted according to the frame, which is shifted to. It is apparent that the pathogenic mutations shift to the +2 frame, whilst the neutral variants shift to the +1 frame. It can be seen that the c-terminal amino acid sequence -PPX is common to all the RTT mutations.

To model the effect of editing the TGA stop codon to TGG (tryptophan) using ABE base editing (shown in FIG. 5(a), a knock-in mouse allele was produced in mouse ES cells. WT JUO9 ES cells were transfected with SpCas9 plasmid pX330 with guide sequence GACCTGAGCCTGAGAGCTCTG cloned into the Bbsl site. The initial G of the guide sequence is not present in the genomic sequence and was added to aid transcription of the guide using the U6 promoter. A single stranded DNA repair template with sequence: AGCAGCAGTGCCTCCTCCCCACCTAAGAAGGAGCACCATCATCACCACCATCACTCAG AGTCCCCAAAGGCGCCCGTGCCACTGCTCCCACCCCATCAGCCCCCCTGGGCCTCAG GACTTGAGCAGCAGCATCTGCAAAGAAGAGAAGATGCCCCGAGGAGGCTCACTGGAAA GCGATGGCTGCCCCAAG was co-transfected with the Cas9 plasmid. The A to G change from the sequence found in the CTD1 mouse is in bold and underlined. ES cell clones were screened for the correct modification of the Mecp2 gene and a correctly targeted clone was used to generate a mouse line by injection into mouse blastocysts. The resultant chimeric founders were bred to establish the CTD1 NS line.

The level of MeCP2 protein in the brains of CTD1 NS hemizygous male mice was measured by western blotting of whole brain extracts FIG. 5(b) (method as described in Guy et al, 2018). The CTD1 NS MeCP2 protein was slightly larger than CTD1, as expected due to the extended tail of missense amino acids, and expressed at levels at or above those in WT littermates (quantified in FIG. 5(c)).

CTD1 NS hemizygous males did not show the RTT-like phenotypes seen in the CTD1 line (and other RTT mouse models) and were indistinguishable from WT littermates when scored weekly FIG. 5(d) as previously described (Guy et al, 2018 and references therein). All CTD1 NS animals survived to one year (CTD1 male hemizygotes median survival 20 weeks).

This mouse model shows that changing the stop codon in a CTD allele to tryptophan using, for example, adenine base editing is likely to prove therapeutic for this class of patients.

A cell line model system was used to test ABE plasmids and guide RNA sequences for adenine editing efficiency. Cell lines with tetracycline-inducible Mecp2 cDNA transgenes FIG. 6(a) were created using the Flp-In™ T-Rex™ system (ThermoFisher). WT and mutant Mecp2 cDNAs (mouse e1 isoform) were cloned into vector pcDNA5/FRT/TO and the resulting plasmids were transfected into Flp-In™ T-Rex™ 293 cell line along with the Flp plasmid pOG44. After selection for recombination of the transgene into the Flp-In locus with HygromycinB, pools of cells were characterised and used for further experiments. Cell lines were induced to express the transgenes using tetracycline and were found by western blotting to reproduce the differences in MeCP2 protein levels seen in mouse knock-in models (as described in Guy et al, 2018) for a range of induction periods and tetracycline concentrations FIG. 6(b). cDNA was made from total RNA preps from induced cells and quantified by real-time qPCR. The differences in mRNA levels seen in knock-in mouse models were also recapitulated in this cellular system FIG. 6(b). In FIG. 6(c) a typical transfection experiment is shown. Cells are transfected with ABE and Guide constructs using Lipofectamine 2000 transfection reagent. After 24 hours the transfection medium is replaced with standard growth medium and cells are then grown for a further 4-5 days before harvesting cell pellets for further analysis by trypsination. For protein or mRNA analysis cells are induced with tetracycline (typically 0.5 g/ml) for 3-24 hours. It should be noted that there was no selection for transfected cells in these experiments, although the transfection efficiency, as estimated by expression of a GFP marker from the ABE plasmids, was high at around 80%. The editing efficiencies are therefore lower than they would be if only transfected cells were considered. FIG. 7 shows results from a typical transfection experiment to determine the editing efficiency of different ABE/guideRNA combinations.

ABE constructs tested were fusions SpG-ABE8 and SpRY-ABE8, formed between a deaminase and nCas9 domain. These constructs consist of adenine deaminase domains as described in Richter et a (2020) (ABE8e) combined with the nCas9 SpG and SpRY variants described in Walton et al (2020). Methods of making fusions are described by Kluesner et al. (2021); Nishida, K. et al. (2016); Komor, A. C., 2016 and Gaudelli N. M. et al. (2017).

Guide sequences with 20 nucleotides homologous to the mouse Mecp2 sequence surrounding the stop codon to be edited were cloned into plasmid pGuide (obtained from Addgene, plasmid #64711, depositor Dr Kiran Musunuru). Guide homology sequences were:
SpG guide 1: CCCCTGAGCCTCAGGACTTG (AGC), A to be edited at guide position 7
SpG guide 2: CTGAGCCTCAGGACTTGAGC (AGC), A to be edited at guide position 4
SpRY guide 5: CCTGAGCCTCAGGACTTGAG (CAG), A to be edited at guide position 5

Guides 1 and 2 can be used by SpG- and SpRY-ABE8e due to their NGN PAM sequence (shown in brackets after the guide sequence). Guide 5 can only be used with SpRY-ABE8e due to the NAN PAM. SpG-Cas9 recognises NGN PAMs and SpRY-ABE8e has a looser PAM specificity recognising NRN, where R=A or G.

As a non-targeting control, the pGuide plasmid with no guide sequence inserted was used in combination with an ABE. This was termed “empty guide” or “g0”.

For genomic DNA analysis, the region surrounding the edited base was amplified by PCR and either the bulk PCR was Sanger sequenced, or amplicons were used for high-throughput sequencing using the Illumina Mi-Seq platform. For bulk sequencing, sequencing traces were analysed using webtool EditR (Kluesner et al, 2018).

Protein levels after editing were measured using western blotting of protein extracts from cell pellets harvested after tetracycline induction FIGS. 7(c), (d). Total intensity of the CTD1 MeCP2 bands (edited and unedited) in each lane was normalised to the histone H3 loading control band. These values were then further normalised with the mean value of the empty guide samples set as 1, FIG. 7(d). Each lane/value represents an independently transfected well of cells.

FIG. 7 (b)-(d) show all combinations of ABE8e and guide plasmids. Five days after transfection cells were induced with tetracycline for 4 hours and then each dish was trypsinised, the cells split into three aliquots and pelleted cells snap frozen and used to prepare genomic DNA, protein or total RNA. Each data point represents an independently transfected dish of cells. When analysing genomic DNA for editing efficiency, it was found that guides 2 and 5 also had significant A to G editing at bystander positions 6 and 9 within the guide sequence. Although these edits do not affect the amino acid sequence of the CTD1 allele as here they are silent mutations, they would make missense edits on the WT allele. Although data on missense neutral variants shows such changes are unlikely to be detrimental to MeCP2 function, it was decided to proceed with guide 1 (and SpG-ABE8e) as this combination gives the highest and cleanest editing. Guide 1 was expected to have the lowest risk of bystander edits as having the on-target edit at position 7 of the guide means that the two downstream As are further from the editing window than with guide 1 (on-target A position 4) or guide 5 (position 5).

The inventors have also split an ABE8e construct into two parts in order to fit them into two AAV vectors (FIG. 8). The use of split intein sequences in the constructs means that the two halves are joined by protein splicing when they are expressed in the same cell, to produce functional full-length protein. AAV is the vector of choice for delivery to brain (initially in mice in our case), but has a size limit less than a single ABE expression construct. The use of two AAV vectors and splitting a nucleic acid into two parts, as achieved above, is described, for example, in Chen (2020). Pairs of split constructs made from both SpG and SpRY ABE8e were tested in combination with SpG guide 1 and compared with the equivalent full-length ABE8es. Editing efficiencies with split ABE8e pairs was comparable to that achieved with the full-length constructs. The split site between amino acids 573 and 574 of SpCas9 was chosen for further work due to the approximately equal sizes of the N- and C-terminal constructs.

### REFERENCES

1. Ross et al. Human Molecular Genetics, Volume 25, Issue 20, 15 Oct. 2016, Pages 4389-4404
2. Krishnaraj et al Human Mutation volume 38, issue 8, 2017)
3. Karczewski, et al. Nature 581, 434-443 (2020)
4. Bebbington et al, Journal of Medical Genetics 2010; 47:242-248
5. Walton et al, Science, Vol 368, Issue 6488, pp 290-296 (2020)
6. Gaudelli et al. Nature Biotechnology volume 38, pages 892-900 (2020)
7. Richter et al Nature Biotechnology volume 38, pages 883-891 (2020)
8. Chen et al; 2022, bioRxiv (doi: https://doi.org/10.1101/2022.08.12.503700)
9. Kleinstiver, B. P. et al, Nature volume 529, pages 490-495 (2016)
10. Slaymaker et al; Science, Vol. 351, Issue 6268, pp 84-88, 2015
11. Vakulskas et al, Nature Medicine volume 24, pages 1216-1224 (2018)
12. Chen et al Small Methods Volume 4, Issue 9, (2020)
13. Grunewald, et al. Nat Biotechnol 37, 1041-1048 (2019)
14. Rees et al., Science Advancesm Vol 5, Issue 5, (2019)
15. Kluesner et al (2021) Nature Communications volume 12, Article number: 2437 (2021)
16. Nishida, K. et al. (2016) Science 16; 353(6305)
17. Komor, A. C. et al Nature 533, 420-424 (2016).
18. Gaudelli, N. M. et al. Nature 551, 464-471 (2017).
19. Erdaki, A. et al. Mol. Cell 73, 714-726 (2019)

1. A base editing construct for editing a mutant MECP2 gene comprising a C-terminal deletion which results in a translation frameshift (n+2) and expression of a truncated MECP2 gene ending -Pro-Pro-Stop, wherein the construct is capable of editing the stop codon in order to permit translational read-through.

2. A method of treating Rett Syndrome in a subject in need thereof, wherein the subject comprises a mutant MECP2 gene comprising a C-terminal deletion which results in a translation frameshift (n+2) and expression of a truncated MECP2 gene ending -Pro-Pro-Stop, wherein the method comprises administering the base editing construct of claim 1 to the subject and wherein the construct is capable of editing the stop codon in order to permit translational read-through.

3. The base editing construct according to claim 1, comprising a base editor that edits an adenine base in a TGA stop codon to another base, such as guanine, or optionally cytosine, or thymine.

4. The base editing construct according to claim 3, wherein the construct comprises a single-guide RNA (sgRNA) to target an adenine base editor (ABE) to the target adenine of the TGA stop codon.

5. The base editing construct according to claim 4, wherein the ABE is ABE8 and derivatives thereof including AB8e and fusions including SpG-ABE8 and SpRY-ABE8.

6. The base editing construct according to claim 4, wherein the sgRNA is 80-150, such as 90-100 nucleotides in length and comprise a sequence of at least 10, at least 15, or at least 20, or 25 contiguous nucleotides that is complementary to a target nucleotide sequence comprising a nucleic acid encoding -Pro-Pro-Stop.

7. The base editing construct according to either of claim 4, wherein the sgRNA comprises the sequence CCCCTGAGCCTCAGGACTTG (AGC); CTGAGCCTCAGGACTTGAGC (AGC), or CCTGAGCCTCAGGACTTGAG (CAG).

8. A nucleic acid construct or constructs encoding the sgRNA and ABE according to claim 4, optionally wherein the nucleic acid construct or constructs is/are provided in an expression vector or vectors.

9. The nucleic acid constructs according to claim 8, wherein the sgRNA and ABE are provided by separate constructs.

10. (canceled)

11. The nucleic acid construct or constructs according to claim 8, wherein the expression vector or vectors is/are a plasmid, phagemid, and/or viral vector or vectors.

12. The nucleic acid construct or constructs according to claim 11, wherein the expression vector comprises an adeno-associated virus (AAV) vector.

13. The nucleic acid construct or constructs according to claim 12, wherein the nucleic acid encoding the ABE is split and expressed by two or more AAV vectors.

14. (canceled)

15. (canceled)

16. (canceled)

17. The method according to claim 2, wherein the base editing construct comprises a base editor that edits an adenine base in a TGA stop codon to another base, such as guanine, or optionally cytosine, or thymine.

18. The method according to claim 17, wherein the base editing construct comprises a single-guide RNA (sgRNA) to target an adenine base editor (ABE) to the target adenine of the TGA stop codon.

19. The method according to claim 18, wherein the ABE is ABE8 and derivatives thereof including AB8e and fusions including SpG-ABE8 and SpRY-ABE8.

20. The method according to claim 18, wherein the sgRNA is 80-150, such as 90-100 nucleotides in length and comprise a sequence of at least 10, at least 15, or at least 20, or 25 contiguous nucleotides that is complementary to a target nucleotide sequence comprising a nucleic acid encoding -Pro-Pro-Stop.

21. The method according to claim 18, wherein the sgRNA comprises the sequence CCCCTGAGCCTCAGGACTTG (AGC); CTGAGCCTCAGGACTTGAGC (AGC), or CCTGAGCCTCAGGACTTGAG (CAG).

22. The method according to claim 2, wherein the base editing construct is provided as an expression vector or vectors.

23. The method according to claim 22, wherein the expression vector or vectors is/are a plasmid, phagemid, and/or viral vector or vectors; optionally, wherein the expression vector comprises an adeno-associated virus (AAV) vector.

24. The method according to claim 23, wherein the ABE is split and expressed by two or more AAV vectors.
